(19) United States
(12) Patent Application Publication
(10) Pub. No.: US 2025/0268688 A1
(43) Pub. Date: Aug. 28, 2025

(54) Robotic Surgical System, Kinematic Mounting Assembly, and Interface Component

(71) Applicant: MAKO Surgical Corp., Weston, FL (US)

(72) Inventors: Jevin Scrivens, Weston, FL (US); Mark Palm, Davie, FL (US)

(73) Assignee: MAKO Surgical Corp., Weston, FL (US)

(21) Appl. No.: 19/064,168

(22) Filed: Feb. 26, 2025

Related U.S. Application Data
(60) Provisional application No. 63/558,706, filed on Feb. 28, 2024.

(51) Int. Cl. A61B 90/50; A61B 34/30

(52) U.S. Cl. CPC A61B 90/50; A61B 34/30; A61B 2034/305

(57) ABSTRACT

A kinematic mounting assembly includes a first mount component having a first and second elongated mount surfaces spaced relative to one another. The kinematic mounting assembly also includes a second mount component having a third and fourth elongated mount surfaces spaced relative to one another. The kinematic mounting assembly further includes an interface component disposed between the first and second mount components. The interface component includes an interface body and a first elongated kinematic coupler and a second elongated kinematic coupler coupled to the interface body. The first elongated kinematic coupler is engageable with the first and third elongated mount surfaces. The second elongated kinematic coupler is engageable with the second and fourth elongated mount surfaces.

### CROSS-REFERENCE TO RELATED APPLICATIONS

The present application claims priority to and all the benefits of U.S. Provisional Patent Application No. 63/558,706, filed on Feb. 28, 2024, which is hereby expressly incorporated herein by reference in its entirety.

### BACKGROUND

### 1. Field of the Invention

The invention relates generally to robotic surgical systems including kinematic mounting assemblies.

### 2. Description of the Related Art

Conventional mounting assemblies known in the art are used to mount surgical components in a robotic surgical system. Often, these mounting assemblies are kinematic such that degrees of freedom of movement are restricted between the surgical components. The surgical components of the mounting assemblies known in the art require either sterilization or, if sterilization is not practical for the particular components of the mounting assemblies, draping with a sterile surgical drape. Often, one of the surgical components being mounted with conventional mounting assemblies is sterilizable and the other of the surgical components being mounted with conventional mounting assemblies requires draping. Once mounted, however, the sterilized surgical component cannot be detached from the draped surgical component without breaking a sterile barrier formed therebetween. Thus, the sterilized surgical component cannot be detached from the draped surgical component during a surgical procedure, and the sterilized surgical component cannot be replaced with another sterilized surgical procedure, limiting the effectiveness of the robotic surgical system in assisting with the surgical procedure.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In one aspect, a kinematic mounting assembly is provided for coupling a first surgical component and a second surgical component, the kinematic mounting assembly comprising: a first mount component couplable to the first surgical component, with the first mount component comprising: a first elongated mount surface, and a second elongated mount surface spaced relative to the first elongated mount surface; a second mount component couplable to the second surgical component, with the second mount component comprising: a third elongated mount surface, and a fourth elongated mount surface spaced relative to the third elongated mount surface; and an interface component disposed between the first mount component and the second mount component, the interface component comprising: an interface body, a first elongated kinematic coupler coupled to the interface body and engageable with the first elongated mount surface and the third elongated mount surface, and a second elongated kinematic coupler coupled to the interface body and engageable with the second elongated mount surface and the fourth elongated mount surface.

In a second aspect, an interface component is provided that is disposable between a first mount component and a second mount component in a kinematic mounting assembly, the interface component comprising: an interface body including a first interface surface configured to interface with the first mount component and a second interface surface configured to interface with the second mount component; a first elongated kinematic coupler having an elongated shape and being coupled to the interface body and moveable relative to the interface body; and a second elongated kinematic coupler having an elongated shape and being coupled to the interface body and moveable relative to the interface body.

In a third aspect, a kinematic mounting assembly for coupling a first surgical component and a second surgical component is provided. The kinematic mounting assembly includes a first mount component couplable to the first surgical component. The first mount component includes a first kinematic mount surface having a first elongated shape and a second kinematic mount surface having a second elongated shape different from the first elongated shape. The first kinematic mount surface and the second kinematic mount surface spaced relative to one another. The kinematic mounting assembly also includes a second mount component couplable to the second surgical component. The second mount component includes a third kinematic mount surface having a third elongated shape and a fourth kinematic mount surface having a fourth elongated shape different from the third elongated shape. The third kinematic mount surface and the fourth kinematic mount surface spaced relative to one another. The kinematic mounting assembly further includes an interface component disposed between the first mount component and the second mount component. The interface component includes a first kinematic coupler having a fifth elongated shape. The first kinematic coupler is engageable with the first kinematic mount surface and the third kinematic mount surface. The interface component also includes a second kinematic coupler having the fifth elongated shape. The second kinematic coupler is engageable with the second kinematic mount surface and the fourth kinematic mount surface.

In a fourth aspect, a mounting system for coupling first and second surgical components is provided. The mounting system includes a first mounting portion coupled to the first surgical component. The first mounting portion includes a first pair of kinematic mounting surfaces having different shapes as one another and spaced relative to one another. The first mounting portion also includes a kinematic post or a kinematic slot. The mounting system also includes a second mounting portion coupled to the second surgical component. The second mounting portion includes a second pair of kinematic mounting surfaces having different shapes as one another and spaced relative to one another. The second mounting portion also includes the other of the kinematic post or the kinematic slot. The mounting system further includes a barrier portion configured to be disposed between the first and second mounting portions. The barrier portion includes a pair of kinematic couplers having identical shapes as one another and spaced relative to one another. When the portions are secured together, the pair of kinematic couplers are configured to engage the first and second pair of kinematic mounting surfaces and the kinematic post is configured to engage the kinematic slot to collectively constrain six degrees of freedom of movement between the first and second surgical components.

In a fifth aspect, a mounting system for coupling first and second surgical components is provided. The mounting system includes a first mounting portion coupled to the first surgical component. The first mounting portion includes a first pair of kinematic mounting surfaces having different shapes as one another and extending approximately parallel to one another. The mounting system also includes a second mounting portion coupled to the second surgical component. The second mounting portion includes a second pair of kinematic mounting surfaces having different shapes as one another and extending approximately parallel to one another. The mounting system further includes a barrier portion configured to be disposed between the first and second mounting portions. The barrier portion includes a pair of kinematic couplers having identical shapes as one another and extending approximately parallel to one another. When the portions are secured together, the pair of kinematic couplers are configured to engage the first and second pair of kinematic mounting surfaces to collectively constrain movement between the first and second surgical components.

According a sixth aspect, a robotic surgical system is provided comprising: a robotic arm having a distal flange; a surgical end effector; and the kinematic mounting assembly of any preceding aspect, with the first mount component fixed to the distal flange of the robotic arm and with the second mount component fixed to the surgical end effector.

In some implementations, the first elongated mount surface extends approximately parallel to the second elongated mount surface, the third elongated mount surface extends approximately parallel to the fourth elongated mount surface, and the first elongated kinematic coupler extends approximately parallel to the second elongated kinematic coupler. In some implementations, the interface body defines a first elongated opening and a second elongated opening spaced from the first elongated opening. The first elongated kinematic coupler may be disposed in the first elongated opening and moveable within the first elongated opening relative to the interface body. The second elongated kinematic coupler may be disposed in the second elongated opening and moveable within the second elongated opening relative to the interface body. In some implementations, the first elongated opening and the second elongated opening are defined approximately parallel to one another. In some implementations, the first elongated kinematic coupler is resiliently fixed to the interface body within the first elongated opening and the second elongated kinematic coupler is resiliently fixed to the interface body within the second elongated opening. In some implementations, engagement of the first elongated kinematic coupler with the first elongated mount surface and the third elongated mount surface, and engagement of the second elongated kinematic coupler with the second elongated mount surface and the fourth elongated mount surface, collectively constrains five degrees of freedom of movement between the first mount component and the second mount component. In some implementations, the first and second elongated kinematic couplers can be integrally formed into or by the interface body (e.g., with or without openings. The first and/or second elongated kinematic couplers may be integrally formed with the interface body. The first and/or second elongated kinematic couplers may be formed of flexible/elastic/resilient material and/or may be coupled to the interface body with flexible/elastic/resilient material that enables the first and/or second elongated kinematic couplers to move relative to the interface body.

In some implementations, the first elongated mount surface has a first elongated shape, the second elongated mount surface has a second elongated shape, and the second elongated shape is different from the first elongated shape. In some implementations, the third elongated mount surface has the first elongated shape and the fourth elongated mount surface has the second elongated shape. In some implementations, the first elongated shape of the first elongated mount surface and the third elongated mount surface are planar such that one rotational degree of freedom of movement is constrained between the first mount component and the second mount component upon engagement of the first elongated kinematic coupler with the first elongated mount surface and the third elongated mount surface. In some implementations, the second elongated shape of the second elongated mount surface and the fourth elongated mount surface is a concave groove. In some implementations, the concave groove is further defined as a V-shaped groove. In some implementations, engagement of the first elongated kinematic coupler with the second elongated mount surface and the fourth elongated mount surface collectively constrains four degrees of freedom of movement between the first mount component and the second mount component. In some implementations, the first elongated kinematic coupler is cylindrical and the second elongated kinematic coupler is cylindrical.

In some implementations, the interface body has a first interface surface facing the first mount component to interface with the first mount component, the interface body has a second interface surface facing the second mount component to interface with the second mount component, the first elongated opening is defined to extend from the first interface surface through the interface body to the second interface surface, and the second elongated opening is defined to extend from the first interface surface through the interface body to the second interface surface.

In some implementations, the first mount component includes one chosen from a kinematic post and a kinematic slot, the second mount component includes the other of the kinematic post and the kinematic slot, and the kinematic post is engageable with the kinematic slot to constrain one degree of freedom of movement between the first mount component and the second mount component. In some implementations, engagement of the first elongated kinematic coupler with the first elongated mount surface and the third elongated mount surface, engagement of the second elongated kinematic coupler with the second elongated mount surface and the fourth elongated mount surface, and engagement of the kinematic post with the kinematic slot collectively constrains six degrees of freedom of movement between the first mount component and the second mount component.

In some implementations, the interface component defines an intermediate kinematic slot engageable with the kinematic post, and the interface component has an intermediate kinematic post engageable with the kinematic slot. In some implementations, the kinematic slot is aligned with the intermediate kinematic slot and the kinematic post is aligned with the intermediate kinematic post. In some implementations, the kinematic post is engageable with the kinematic slot without direct contact therebetween.

In some implementations, the first mount component defines a first notch and a second notch spaced from the first notch, and the interface component includes a first resilient insert disposable in the first notch and a second resilient insert disposable in the second notch such that the interface component is detachably coupled to the first mount component. In some implementations, the first resilient insert has a first proximate portion and a first distal portion extending distally away from the first proximate portion, and the second resilient insert has a second proximate portion and a second distal portion extending away from the second proximate portion. In some implementations, the first distal portion is angled to extend toward the first mount component and the second distal portion is angled to extend toward the first mount component. In some implementations, the first resilient insert includes a first tab extending from the first proximate portion toward the first mount component and into the first notch, with the first tab having a first lip extending proximally from the first tab and engageable with the first mount component to prevent the first resilient insert from moving away from the first mount component, and the second resilient insert includes a second tab extending from the second proximate portion toward the second mount component and into the second notch, with the second tab having a second lip extending proximally from the second tab and engageable with the first mount component to prevent the second resilient insert from moving away from the first mount component.

In some implementations, the first distal portion of the first resilient insert is moveable toward the second mount component to move the first resilient insert out of the first notch and the second distal portion of the second resilient insert is moveable toward the second mount component to move the second resilient insert out of the second notch, thus detaching the interface component from the first mount component. In some implementations, the second mount component defines a first bore and a second bore, the first proximate portion of the first resilient insert defines a first threaded bore aligned with the first bore of the second mount component, the second proximate portion of the second resilient insert defines a second threaded bore aligned with the second bore of the second mount component, and the kinematic mounting assembly further includes a first fastener disposed in the first bore and the first threaded bore and a second fastener disposed in the second bore and the second threaded bore to fix the second mount component to the interface component.

In some implementations, the kinematic mounting assembly further includes a drape configured to cooperate with the interface component to form a sterile barrier to the first mount component. In some implementations, the interface component and the drape permit the second mount component to be detached from the interface component without breaking the sterile barrier to the first mount component. In some implementations, the first mount component and the interface component are together configured to permit a drape to be positioned such that a sterile barrier to the first mount component is formed by the interface component and the drape. In some implementations, the second mount component is detachable from the interface component without breaking a sterile barrier to the first mount component. In some implementations, the interface body includes a central hub defining the first elongated opening and the second elongated opening and an outer flange disposed about the central hub, with the outer flange configured to cooperate with a drape such that the interface component and the drape form a sterile barrier to the first mount component.

In some implementations, the first surgical component is a distal flange of a robotic arm, and the second surgical component is a surgical end effector. In some implementations, a robotic surgical system is provided, with the robotic surgical system including a robotic arm having a distal flange, a surgical end effector, and the kinematic mounting assembly of any of the aspects and implementations as described herein, with the first mount component fixed to the distal flange of the robotic arm and the second mount component fixed to the surgical end effector.

Any of the above aspects can be combined in full or in part. Any features of the above aspects can be combined in full or in part. Any of the above implementations can be combined, in full or in part, with any other aspect. Any of the above implementations can be combined with any other implementation whether for the same aspect or different aspect.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a robotic surgical system, according to one implementation;

FIG. 2 is a perspective view of a kinematic mount assembly, according to one implementation;

FIG. 3 is an exploded view of the kinematic mounting assembly of FIG. 2;

FIG. 4 is another perspective view of the kinematic mounting assembly

of FIG. 2;

FIG. 5 is a cross-sectional view of the kinematic mounting assembly of

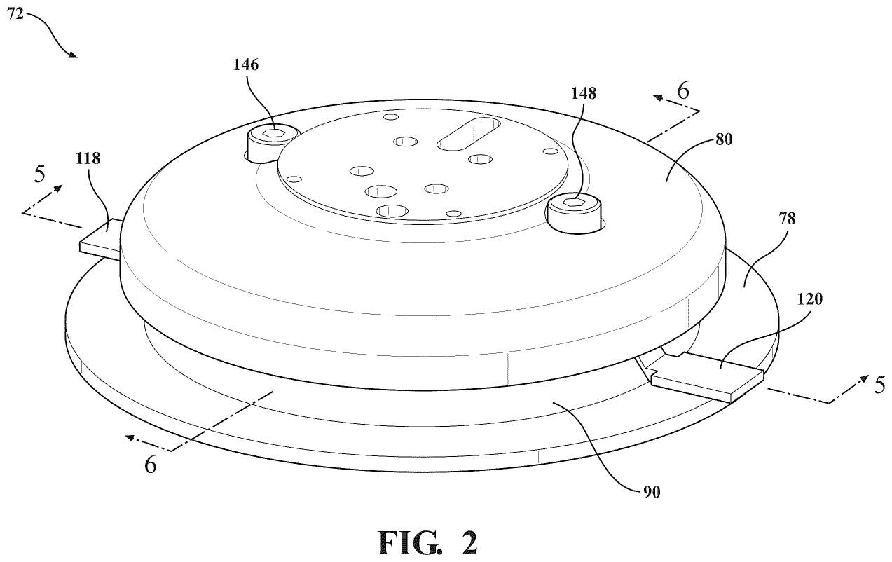

FIG. 2 taken along line 5-5; and

FIG. 6 is another cross-sectional view of the kinematic mounting assembly of FIG. 2 taken along line 6-6.

### DETAILED DESCRIPTION OF THE INVENTION

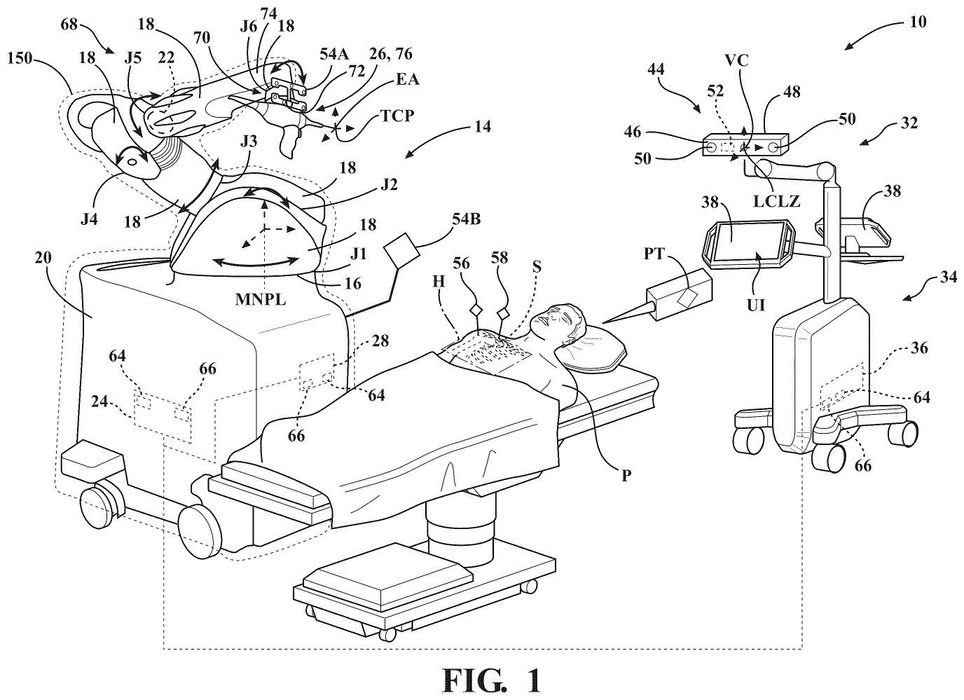

With reference to the Figures, wherein like numerals indicate like parts throughout the several views, a robotic surgical system 10 is illustrated. The robotic surgical system 10 is useful for treating a surgical site or anatomical volume of a patient P, such as treating bone or soft tissue. In FIG. 1, the patient P is undergoing a surgical procedure. The surgical procedure may involve tissue removal or other forms of treatment. Treatment may include cutting, coagulating, lesioning the tissue, other in-situ tissue treatments, or the like. In some examples, the surgical procedure involves shoulder replacement surgery, partial or total knee or hip replacement surgery, spine surgery, or ankle surgery. In some examples, the system 10 is designed to cut away material to be replaced by surgical implants, such as shoulder implants, partial or total knee implants, hip implants, spine implants, or ankle implants. In FIG. 1, the system 10 is shown being employed to prepare the humerus H and/or a glenoid cavity of a scapula S to receive shoulder implants. Examples of shoulder implants, and methods of implanting them, are shown in U.S. Patent Publication No. 2022/0039898, filed on Aug. 4, 2021, entitled, “Robotic Surgical System Including a Coupler for Connecting a Tool to a Manipulator and Methods of Using the Coupler,” U.S. Pat. No. 11,432,945, filed on Nov. 6, 2018, entitled, “Robotic System For Shoulder Arthroplasty Using Stemless Implant Components,” and U.S. Pat. No. 11,173,048, filed on Nov. 6, 2018, entitled, “Robotic System For Shoulder Arthroplasty Using Stemless Implant Components,” the disclosures of which are hereby incorporated herein by reference. The system 10 and techniques disclosed herein may be used to perform other procedures, surgical or non-surgical, or may be used in industrial applications or other applications where robotic systems are utilized.

The system 10 includes a manipulator 14. The manipulator 14 has a base 16 and plurality of links 18. A manipulator cart 20 can support the manipulator 14 such that the manipulator 14 is fixed to the manipulator cart 20. In other examples, the manipulator 14 can be mounted to a surgical patient table. The links 18 collectively form one or more arms or linkages of the manipulator 14 with adjacent links being connected by joints. The manipulator 14 may have a serial, robotic arm configuration (as shown in FIG. 1), a parallel, robotic arm configuration, or any other suitable manipulator configuration. In other examples, more than one manipulator 14 may be utilized in a multiple arm configuration.

In the example shown in FIG. 1, the manipulator 14 comprises a plurality of joints J1-J6 and a plurality of joint encoders 22 located at the joints J1-J6 for determining position data (e.g., rotation angles) of the joints J1-J6. For simplicity, only one joint encoder 22 is illustrated in FIG. 1, although other joint encoders 22 may be similarly illustrated. The manipulator 14 according to one example has six joints J1-J6 implementing at least six-degrees of freedom (DOF) for the manipulator 14. However, the manipulator 14 may have any number of degrees of freedom and may have any suitable number of joints J and may have redundant joints.

The manipulator 14 need not require joint encoders 22 but may alternatively, or additionally, utilize motor encoders present on motors at each joint J. Also, the manipulator 14 need not require rotary joints, but may alternatively, or additionally, utilize one or more prismatic joints. Any suitable combination of joint types are contemplated.

The base 16 of the manipulator 14 is generally a portion of the manipulator 14 that provides a fixed reference coordinate system for other components of the manipulator 14 or the system 10 in general. Generally, the origin of a manipulator coordinate system MNPL is defined at the fixed reference of the base 16. The base 16 may be defined with respect to any suitable portion of the manipulator 14, such as one or more of the links 18. Alternatively, or additionally, the base 16 may be defined with respect to the manipulator cart 20, such as where the manipulator 14 is physically attached to the cart 20. In one example, the base 16 is defined at an intersection of the axes of joints J1 and J2. Thus, although joints J1 and J2 are moving components in reality, the intersection of the axes of joints J1 and J2 is nevertheless a virtual fixed reference pose, which provides both a fixed position and orientation reference and which does not move relative to the manipulator 14 and/or manipulator cart 20. In other examples, the manipulator 14 can be a hand-held manipulator where the base 16 is a base portion of a surgical end effector 26, such as a tool (e.g., a portion held free-hand by the user) and the tool tip is movable relative to the base portion. The base portion has a reference coordinate system that is tracked and the tool tip has a tool tip coordinate system that is tracked relative to the reference coordinate system.

The manipulator 14 and/or manipulator cart 20 house a manipulator controller 24, or other type of control unit. The manipulator controller 24 may comprise one or more computers, or any other suitable form of controller that directs the motion of the manipulator 14. The manipulator controller 24 may have a central processing unit (CPU) and/or other processors, memory (not shown), and storage (not shown). The manipulator controller 24 is loaded with software as described below. The processors could include one or more processors to control operation of the manipulator 14. The processors can be any type of microprocessor, multi-processor, and/or multi-core processing system. The manipulator controller 24 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor. The manipulator 14 may also comprise a user interface UI with one or more displays and/or input devices (e.g., push buttons, keyboard, mouse, microphone (voice-activation), gesture control devices, touchscreens, etc.).

A surgical end effector 26, such as a surgical tool 26 as illustrated in FIG. 1, couples to the manipulator 14 and is movable relative to the base 16 to interact with the anatomy in certain modes. More specifically, the manipulator 14 may be referred to as a robotic arm 68 having a distal flange 70, and the surgical end effector 26 may be mounted to the distal flange 70 of the robotic arm 68. The tool 26 is or forms part of an end effector supported by the manipulator 14 in certain embodiments. The tool 26 may be grasped by the user. One possible arrangement of the manipulator 14 and the tool 26 is described in U.S. Pat. No. 9,119,655, entitled, “Surgical Manipulator Capable Of Controlling A Surgical Tool In Multiple Modes,” filed on Aug. 2, 2013, the disclosure of which is hereby incorporated herein by reference. The manipulator 14 and the tool 26 may be arranged in alternative configurations. The tool 26 can be like that shown in U.S. Pat. No. 9,566,121, filed on Mar. 15, 2014, entitled, “End Effector Of A Surgical Robotic Manipulator,” hereby incorporated herein by reference.

The tool 26 includes an energy applicator EA designed to contact and remove the tissue of the patient P at the surgical site. In one example, the energy applicator EA is a burr. The burr may be substantially spherical and comprise a spherical center, radius (r) and diameter. Alternatively, the energy applicator EA may be a drill bit, a saw blade, an ultrasonic vibrating tip, or the like. In some versions, the tool 26 includes non-motorized accessories such as a probe, a retractor, a cutting guide, or the like. The tool 26 and/or energy applicator EA/accessory may comprise any geometric feature, e.g., perimeter, circumference, radius, diameter, width, length, volume, area, surface/plane, range of motion envelope (along any one or more axes), etc. The geometric feature may be considered to determine how to locate the tool 26 relative to the tissue at the surgical site to perform the desired treatment. In some of the embodiments described herein, a spherical burr having a tool center point (TCP) will be described for convenience and ease of illustration but is not intended to limit the tool 26 to any particular form. The tool 26 may include a tool driver 26 that houses any driving motor for the energy applicator EA, e.g., to drive saw blade oscillation, burr rotation, drill rotation, etc.

The tool 26 may comprise a tool controller 28 to control operation of the tool 26, such as to control power to the tool (e.g., to a rotary, driving motor of the tool 26), control movement of the tool 26, control irrigation/aspiration of the tool 26, and/or the like. The tool controller 28 may be in communication with the manipulator controller 24 or other components. The tool 26 may also comprise a user interface UI with one or more displays and/or input devices (e.g., push buttons, keyboard, mouse, microphone (voice-activation), gesture control devices, touchscreens, etc.). The manipulator controller 24 controls a state (e.g., position and/or orientation) of the tool 26 (e.g., the TCP) with respect to a coordinate system, such as the manipulator coordinate system MNPL. The manipulator controller 24 can control (linear or angular) velocity, acceleration, or other derivatives of motion of the tool 26.

The tool center point (TCP), in one example, is a predetermined reference point or coordinate system defined at the energy applicator EA. The TCP has a known, or able to be calculated (i.e., not necessarily static), pose relative to other coordinate systems. The geometry of the energy applicator EA is known in or defined relative to a TCP coordinate system. The TCP may be located at the spherical center of the burr of the tool 26 such that only one point is tracked. The TCP may be defined in various ways depending on the configuration of the energy applicator EA. The manipulator 14 could employ the joint/motor encoders, or any other non-encoder position sensing method, to enable a pose of the TCP to be determined. The manipulator 14 may use joint measurements to determine TCP pose and/or could employ techniques to measure TCP pose directly. The control of the tool 26 is not limited to a center point. For example, any suitable primitives, meshes, etc., can be used to represent the tool 26.

The system 10 further includes a navigation system 32. One example of the navigation system 32 is described in U.S. Pat. No. 9,008,757, filed on Sep. 24, 2013, entitled, “Navigation System Including Optical And Non-Optical Sensors,” hereby incorporated herein by reference. The navigation system 32 tracks movement of various objects. Such objects include, for example, the manipulator 14, the tool 26 and the anatomy, e.g., the humerus H and scapula S. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformations.

The navigation system 32 includes a cart assembly 34 that houses a navigation controller 36, and/or other types of control units. A navigation user interface UI is in operative communication with the navigation controller 36. The navigation user interface includes one or more displays 38. The navigation system 32 is capable of displaying a graphical representation of the relative states of the tracked objects to the user using the one or more displays 38. The navigation user interface UI further comprises one or more input devices to input information into the navigation controller 36 or otherwise to select/control certain aspects of the navigation controller 36. Such input devices include interactive touchscreen displays. However, the input devices may include any one or more of push buttons, a keyboard, a mouse, a microphone (voice-activation), gesture control devices, and the like.

The navigation system 32 also includes a navigation localizer 44 coupled to the navigation controller 36. In one example, the localizer 44 is an optical localizer and includes a camera unit 46. The camera unit 46 has an outer casing 48 that houses one or more optical sensors 50. The localizer 44 may include its own localizer controller 52 and may further include a video camera VC.

The navigation system 32 includes one or more surgical tracker assemblies. In one example, the surgical tracker assemblies include a pointer tracker PT, a surgical tracker assembly 54A for the tool 26, a surgical tracker assembly 54B for the manipulator 14, one or more surgical tracker assemblies 56, 58 for the patient, including surgical tracker 56 for the patient and surgical tracker assembly 58 for the patient. In the illustrated example of FIG. 1, the surgical tracker assembly 54A is fixed with respect to the tool 26, the surgical tracker assembly 56 is firmly affixed to the humerus H of the patient P, and the surgical tracker assembly 58 is firmly affixed to the scapula S of the patient P. In this example, the surgical tracker assemblies 56, 58 for the patient are firmly affixed to sections of bone. The pointer tracker PT is firmly affixed to a pointer used for registering the anatomy to the localizer coordinate system LCLZ. The surgical tracker assembly 54B may be affixed to any suitable component of the manipulator 14, in addition to, or other than the tool 26, such as the base 16, the cart 20, or any one or more links 18 of the manipulator 14. The surgical tracker assemblies 54A, 54B, 56, 58, PT may be fixed to their respective components in any suitable manner. For example, the trackers may be rigidly fixed, flexibly connected (optical fiber), or not physically connected at all (ultrasound), as long as there is a suitable (supplemental) way to determine the relationship (measurement) of that respective tracker to the object that it is associated with. In some cases, only one of the surgical tracker assemblies 54A, 54B for the tool 26 and the manipulator 14 are used, or both the surgical tracker assemblies 54A, 54B for the tool 26 and the manipulator 14 may be used.

In the illustrated embodiment, the surgical tracker assemblies 54A, 54B, 56, 58, PT are passive trackers. Accordingly, each surgical tracker assembly 54A, 54B, 56, 58, PT has at least three passive tracking elements, fiducials, or markers M, such as reflectors, for reflecting light from the localizer 44 back to the optical sensors 50. In other embodiments, the surgical tracker assemblies 54A, 54B, 56, 58, PT are active trackers and may have light emitting diodes or LEDs transmitting light, such as infrared light to the optical sensors 50. Based on the received optical signals, navigation controller 36 generates data indicating the relative positions and orientations of the surgical tracker assemblies 54A, 54B, 56, 58, PT relative to the localizer 44 using conventional triangulation techniques. In some cases, more or fewer markers may be employed. For instance, in cases in which the object being tracked is rotatable about a line, two markers can be used to determine an orientation of the line by measuring positions of the markers at various locations about the line. It should be appreciated that the localizer 44 and surgical tracker assemblies 54A, 54B, 56, 58, PT, although described above as utilizing optical tracking techniques, could alternatively, or additionally, utilize other tracking modalities to track the objects, such as electromagnetic tracking, radio frequency tracking, inertial tracking, ultrasound-based tracking, fiber-optic tracking, machine-vision tracking, combinations thereof, and the like.

The localizer 44 tracks the surgical tracker assemblies 54A, 54B, 56, 58, PT to determine a state of each of the surgical tracker assemblies 54A, 54B, 56, 58, PT, which correspond respectively to the state of the object respectively attached thereto. The localizer 44 provides the state of the surgical tracker assemblies 54A, 54B, 56, 58, PT to the navigation controller 36. In one example, the navigation controller 36 determines and communicates the state of the surgical tracker assemblies 54A, 54B, 56, 58, PT to the manipulator controller 24. As used herein, the state of an object includes, but is not limited to, data that defines the position and/or orientation of the tracked object or equivalents/derivatives of the position and/or orientation. For example, the state may be a pose of the object, and may include linear velocity data, and/or angular velocity data, and the like.

The navigation controller 36 may comprise one or more computers, or any other suitable form of controller. Navigation controller 36 has a central processing unit (CPU) and/or other processors, memory (not shown), and storage (not shown). The processors can be any type of processor, microprocessor or multi-processor system. The navigation controller 36 is loaded with software. The software, for example, converts the signals received from the localizer 44 into data representative of the position and orientation of the objects being tracked. The navigation controller 36 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor.

In operation, for certain surgical tasks, the user manually manipulates (e.g., moves or causes the movement of) the tool 26 to perform the surgical procedure on the patient, such as drilling, cutting, sawing, reaming, implant installation, and the like. As the user manipulates the tool 26, the navigation system 32 tracks the location of the tool 26 and/or the manipulator 14 and provides haptic feedback (e.g., force feedback) to the user to limit the user's ability to move (or cause movement of) the tool 26 beyond one or more predefined virtual boundaries that are registered (or mapped) to the patient's anatomy, which results in highly accurate and repeatable drilling, cutting, sawing, reaming, and/or implant placement.

In some embodiments, the manipulator 14 operates in a passive manner and provides haptic feedback when the surgeon attempts to move the tool 26 beyond the virtual boundary. The haptic feedback (e.g., a form of stereotactic feedback) is generated by one or more actuators (e.g., joint motors) of the manipulator 14 and transmitted to the user via a flexible transmission, such as a cable drive transmission. When the manipulator 14 is not providing haptic feedback, the manipulator 14 is freely moveable by the user. In some embodiments, like that shown in U.S. Pat. No. 9,566,122, incorporated herein by reference, the manipulator 14 is manipulated by the user in a similar manner, but the manipulator 14 operates in an active manner. For instance, the user applies force to the tool 26, which is measured by a force/torque sensor S, and the manipulator 14 emulates the user's desired movement based on measurements from the force/torque sensor S. For other surgical tasks, the manipulator 14 may operate autonomously.

The system 10 includes a control system that comprises, among other components, the manipulator controller 24, the navigation controller 36, and the tool controller 28. The control system further includes one or more software programs and software modules. The software modules may be part of the program or programs that operate on the manipulator controller 24, navigation controller 36, tool controller 28, or any combination thereof, to process data to assist with control of the system 10. The software programs and/or modules include computer readable instructions stored in non-transitory memory 64 on the manipulator controller 24, navigation controller 36, tool controller 28, or a combination thereof, to be executed by one or more processors 66 of the controllers 24, 28, 36. The memory 64 may be any suitable configuration of memory, such as RAM, non-volatile memory, etc., and may be implemented locally or from a remote database. Additionally, software modules for prompting and/or communicating with the user may form part of the program or programs and may include instructions stored in memory 64 on the manipulator controller 24, navigation controller 36, tool controller 28, or any combination thereof. The user may interact with any of the input devices of the navigation user interface UI or other user interface UI to communicate with the software modules. The user interface software may run on a separate device from the manipulator controller 24, navigation controller 36, and/or tool controller 28.

The control system may comprise any suitable configuration of input, output, and processing devices suitable for carrying out the functions and methods described herein. The control system may comprise the manipulator controller 24, the navigation controller 36, or the tool controller 28, or any combination thereof, or may comprise only one of these controllers. These controllers may communicate wirelessly, via a bus as shown in FIG. 2, or otherwise. The control system may also be referred to as a controller. The control system may comprise one or more microcontrollers, field programmable gate arrays, systems on a chip, discrete circuitry, sensors, displays, user interfaces, indicators, and/or other suitable hardware, software, or firmware that is capable of carrying out the functions described herein.

The manipulator controller 24 and/or the navigation controller 36 track the state of the tool 26 relative to the anatomy and the virtual boundaries. In one example, the state of the TCP is measured relative to the virtual boundaries for purposes of determining haptic forces to be applied to a virtual rigid body model via a virtual simulation so that the tool 26 remains in a desired positional relationship to the virtual boundaries (e.g., not moved beyond them, kept within them, etc.). The results of the virtual simulation are commanded to the manipulator 14.

In some implementations, using the navigation system 32, the pose of the tool 26 can be determined by tracking the location of the base 16 and the associated manipulator coordinate system MNPL via the surgical tracker assembly 54B for the manipulator 14 and calculating the pose of the tool 26 based on joint encoder data from the joint encoders 22 (and/or motor encoders) at the joints J1-J6 (using kinematic data) and based on a known geometric relationship between the tool 26 and the manipulator 14. Ultimately, the localizer 44 and the surgical tracker assemblies 54A, 54B, 56, 58, PT enable the determination of the pose of the tool 26 and the patient's anatomy so the navigation system 32 knows the relative relationship between the tool 26 and the patient's anatomy. However, in some cases, the surgical tracker assembly 54B for the manipulator 14 may be out of view of the localizer 44, or the surgical tracker assembly 54B for the manipulator 14 may not be used. Line-of-sight between one or more of the sensors 50 and the surgical tracker assembly 54B for the manipulator 14 may be obstructed such that movement of the tool 26 cannot be reliably tracked solely using the surgical tracker assembly 54B for the manipulator 14 and encoder data. In this case, the surgical tracker assembly 54A for the tool 26 can be employed to track movement of the tool 26, i.e., the surgical tracker assembly 54A for the tool 26 is detected by the localizer 44 to determine a pose of the tool 26 (e.g., of the TCP coordinate system of the tool 26).

The surgical end effector 26 may be coupled to the robotic arm 68 (e.g., to the manipulator 14), particularly to the distal flange 70 of the robotic arm 68, through a kinematic mounting assembly 72. The kinematic mounting assembly 72 may releasably connect the surgical end effector 26 to interchange different energy applicators EA. The kinematic mounting assembly 72 may releasably hold the energy applicator EA.

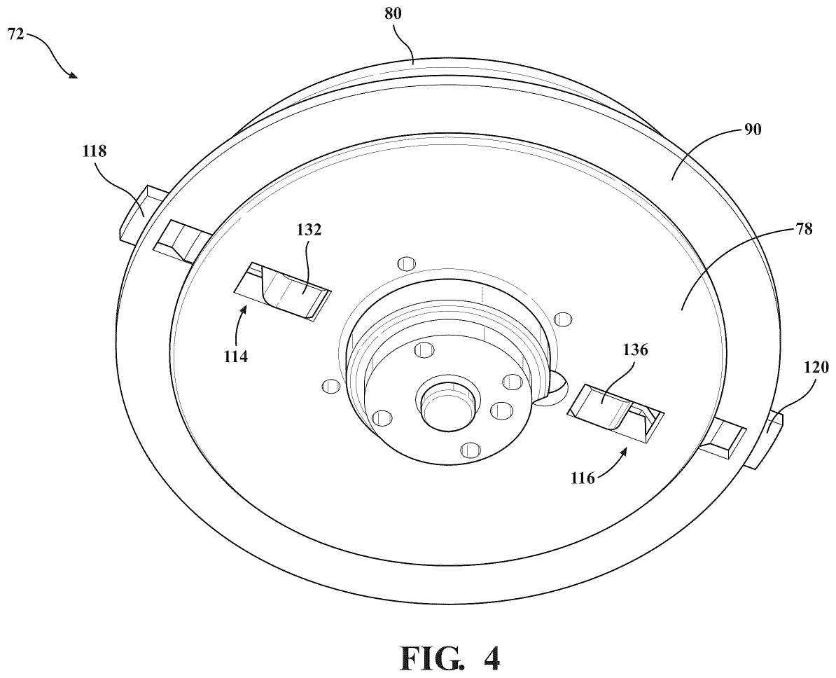

With reference to FIGS. 1-6, wherein like numerals indicate like parts through the several views, the kinematic mounting assembly 72 is provided. In one aspect, the kinematic mounting assembly 72 is for coupling a first surgical component 74 and a second surgical component 76. Although not required, in one implementation as shown in FIG. 1, the first surgical component 74 may be the distal flange 70 of the robotic arm 68, and the second surgical component 76 may be the surgical end effector 26. The robotic surgical system 10, therefore, may include the robotic arm 68 having the distal flange 70, the surgical end effector 26, and the kinematic mounting assembly 72 as described herein. More specifically, a first mount component 78 may be fixed to the distal flange 70 of the robotic arm 68 and a second mount component 80 may be fixed to the surgical end effector 26. In this way, the kinematic mounting assembly 72 may mount the surgical end effector 26 relative to the distal flange 70 of the robotic arm 68. It is to be appreciated, however, that the kinematic mounting assembly 72 is also suitable for mounting a variety of different surgical components and is not limited to the exemplary implementation as disclosed and described herein.

In one aspect, as shown generally throughout FIGS. 2-6, the kinematic mounting assembly 72 includes a first mount component 78 couplable to the first surgical component 74. The first mount component 78 includes a first elongated mount surface 82 and a second elongated mount surface 84 spaced relative to the first elongated mount surface 82. The first elongated mount surface 82 and the second elongated mount surface 84 may extend approximately parallel to one another, such as but not limited to, angularly within about 10% of parallel. The kinematic mounting assembly 72 also includes a second mount component 80 couplable to the second surgical component 76. The second mount component 80 includes a third elongated mount surface 86 and a fourth elongated mount surface 88 spaced relative to the third elongated mount surface 86. The third elongated mount surface 86 and the fourth elongated mount surface 88 may extend approximately parallel to one another, such as but not limited to, angularly within about 10% of parallel. The kinematic mounting assembly 72 further includes an interface component 90 disposed between the first mount component 78 and the second mount component 80.

The interface component 90 includes a first elongated kinematic coupler 98 and a second elongated kinematic coupler 100. The first elongated kinematic coupler 98 is engageable with the first elongated mount surface 82 and the third elongated mount surface 86. The second elongated kinematic coupler 100 is engageable with the second elongated mount surface 84 and the fourth elongated mount surface 88. The first elongated kinematic coupler 98 and the second elongated kinematic coupler 100 may extend approximately parallel to one another, such as but not limited to, angularly within about 10% of parallel. Moreover, the first elongated kinematic coupler 98 and the second elongated kinematic coupler 100 may be co-planar with one another.

In one implementation, the interface component 90 includes an interface body 92. The interface body 92 can define a first elongated opening 94 and a second elongated opening 96. Although not required, the first elongated opening 94 and the second elongated opening 96 may be defined to extend approximately parallel to one another, such as but not limited to, angularly within about 10% of parallel. When the openings 94, 96 are provided, the first elongated kinematic coupler 98 can be disposed in the first elongated opening 94 and the second elongated kinematic coupler 100 can be disposed in the second elongated opening 96.

In one implementation, the first elongated kinematic coupler 98 is moveable within the first elongated opening 94 relative to the interface body 92 and the second elongated kinematic coupler 100 is moveable within the second elongated opening 96 relative to the interface body 92. In other words, the first elongated kinematic coupler 98 need not be immovably fixed to the interface body 92 and the second elongated kinematic coupler 100 need not be immovably fixed to the interface body 92. In the implementations where the first elongated kinematic coupler 98 and the second elongated kinematic coupler 100 are moveable within the first elongated opening 94 and the second elongated opening 96, respectively, the first elongated kinematic coupler 98 is better able to engage the first elongated mount surface 82 and the third elongated mount surface 86 and the second elongated kinematic coupler 100 is better able to engage the second elongated mount surface 84 and the fourth elongated mount surface 88.

Although not required, the first elongated kinematic coupler 98 may be resiliently fixed to the interface body 92 within the first elongated opening 94. Moreover, the second elongated kinematic coupler 100 may be resiliently fixed to the interface body 92 within the second elongated opening 96. In non-limiting examples, the first elongated kinematic coupler 98 may be resiliently fixed to the interface body through a biasing member such as a spring, through rubber such as being embedded in rubber, or the like, and the second elongated kinematic coupler 100 may be resilient fixed to the interface body 92 through a biasing member such as a spring, through rubber such as being embedded in rubber, or the like. In this way, the first elongated kinematic coupler 98 and the second elongated kinematic coupler 100 may be said to float within the first elongated opening 94 and the second elongated opening 96, respectively, with some spacing between the first elongated kinematic coupler 98 and the interface body 92 and with some spacing between the second elongated kinematic coupler 100 and the interface body 92.

In yet another example, the first and second elongated kinematic couplers 98, 100 can be integrally formed into or by the interface body 92 (e.g., with or without openings 94, 96). In such instances, the first and second elongated kinematic couplers 98, 100 may comprise flexible/elastic/resilient material or may be coupled to flexible/elastic/resilient material that enables the first and second elongated kinematic couplers 98, 100 to move relative to the interface body 92.

Engagement of the first elongated kinematic coupler 98 with the first elongated mount surface 82 and the third elongated mount surface 86, and engagement of the second elongated kinematic coupler 100 with the second elongated mount surface 84 and the fourth elongated mount surface 88, may collectively constrain five degrees of freedom of movement between the first mount component 78 and the second mount component 80. The five degrees of freedom of movement may be any five degrees of the following: up/down, forward/back, left/right, pitch, roll, and yaw.

It is to be appreciated that the engagement of the first elongated kinematic coupler 98 with the first elongated mount surface 82 and the third elongated mount surface 86, as well as engagement of the second elongated kinematic coupler 100 with the second elongated mount surface 84 and the fourth elongated mount surface 88, may results in more than one constraint on a particular degree of freedom or on more than one particular degrees of freedom. Although theoretically redundant, such redundant constraint(s) has been found to only increase the robustness of the kinematic mounting between the first mount component 78 and the second mount component 80. Moreover, such redundant constraint(s) are particularly advantageous for real components subject to load. Such redundant constraints are also less of a concern from a practical standpoint due to the relatively tight tolerances able to be machined with modern equipment. In a non-limiting example, a single machining step may be employed to reduce the tolerances of various components of the kinematic mounting assembly 72 and thus permit redundant constraints on a particular degree(s) of freedom.

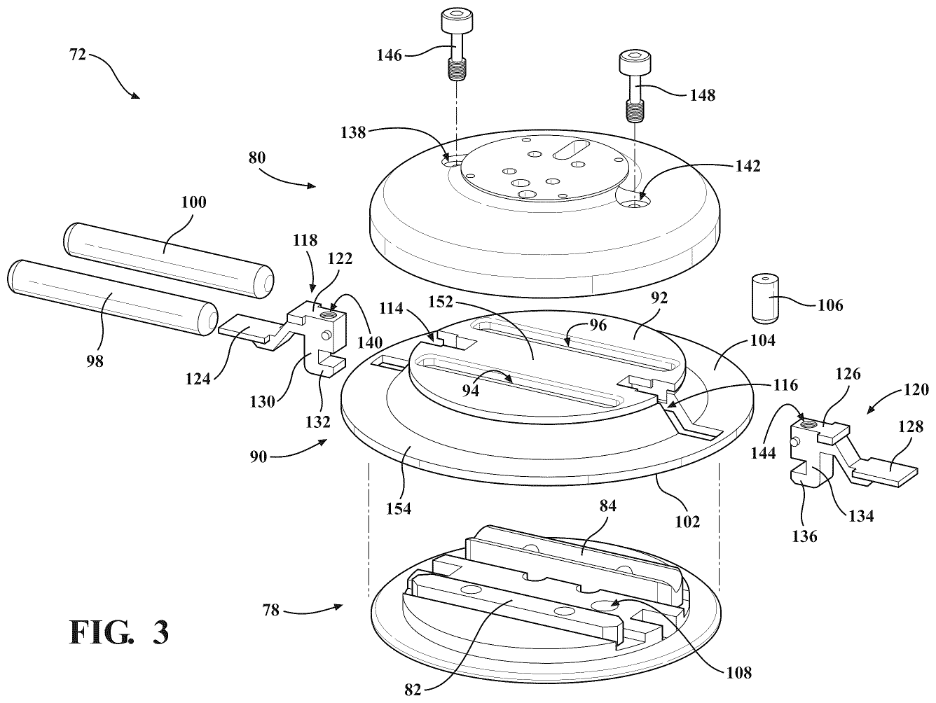

The first elongated mount surface 82 has a first elongated shape, the second elongated mount surface 84 has a second elongated shape, and the second elongated shape may be different from the first elongated shape, as shown in FIGS. 3 and 6. The first elongated shape being different from the second elongated shape may advantageously result in different degrees of freedom of movement being constrained. It is also to be appreciated that the first elongated shape and the second elongated shape may refer to cross-sectional shapes which are extended along the respective elongated mount surfaces.

The third elongated mount surface 86 may have the first elongated shape. In other words, the third elongated mount surface 86 may have the same shape as the first elongated mount surface 82. The fourth elongated mount surface 88 may have the second elongated shape. In other words, the fourth elongated mount surface 88 may have the same shape as the second elongated mount surface 84. As such, the same degrees of freedom of movement are constrained between the first elongated mount surface 82 and the third elongated mount surface 86, and the same degrees of freedom of movement are constrained between the second elongated mount surface 84 and the fourth elongated mount surface 88.

Although not required, the first elongated shape of the first elongated mount surface 82 and the third elongated mount surface 86 may be planar such that one degree of freedom of movement is constrained between the first mount component 78 and the second mount component 80 upon engagement of the first elongated kinematic coupler 98 with the first elongated mount surface 82 and the third elongated mount surface 86. With reference to the view of FIG. 6, the one degree of freedom of movement constrained by engagement of the first elongated kinematic coupler 98 with the first elongated mount surface 82 and the third elongated mount surface 86 may be understood as one rotational degree of freedom. This one degree of rotation may be defined as rotation (i.e., pitch) about an X-axis extending horizontally (left to right) through the first elongated kinematic coupler 98.

The second elongated shape of the second elongated mount surface 84 and the fourth elongated mount surface 88 may be a concave groove. In a non-limiting example, the concave groove is further defined as a V-shaped groove, as shown in FIGS. 3 and 6. In other non-limiting examples, the concave groove may be U-shaped, may be an arch such as a gothic arch, may be tapered, or may be rectangular in shape. Engagement of the first elongated kinematic coupler 98 with the second elongated mount surface 84 and the fourth elongated mount surface 88 may collectively constrain four degrees of freedom of movement between the first mount component 78 and the second mount component 80. The four degrees of freedom of movement constrained by engagement of the second elongated kinematic coupler 100 with the second elongated mount surface 84 and the fourth elongated mount surface 88 may be understood as two degrees of rotational freedom and two translational degrees of freedom. With reference to the view of FIG. 6, the two translational degrees of freedom may be, e.g., (1) Z-axis (up and down) and X-axis (left to right), and the two rotational degrees of freedom may be, e.g., rotation about the Z-axis (yaw) and rotation about the X-axis (pitch) or rotation (e.g., roll) about the Y-axis (in and out the page).

The first elongated kinematic coupler 98 may be cylindrical and the second elongated kinematic coupler 100 may be cylindrical. In other words, the first elongated kinematic coupler 98 may be circular in cross-section and the second elongated kinematic coupler 100 may be circular in cross-section. The interface body 92 has a first interface surface 102 facing the first mount component 78 to interface with the first mount component 78, and the interface body 92 has a second interface surface 104 facing the second mount component 80 to interface with the second mount component 80. Although not required, the first elongated opening 94 may be defined to extend from the first interface surface 102 through the interface body 92 to the second interface surface 104, and the second elongated opening 96 may be defined to extend from the first interface surface 102 through the interface body 92 to the second interface surface 104. In other words, the first elongated opening 94 and the second elongated opening 96 may be defined to extend completely through the interface body 92. It is to be appreciated that the interface body 92 may be formed as a plate and may also be referred to as an interface plate, in some implementations. Moreover, the first elongated opening 94 and the second elongated opening 96 may be defined to extend completely through the interface body 92 in a direction approximately perpendicular to the interfaces between the first mount component 78 and the interface component 90 and between the second mount component 80 and the interface component 90.

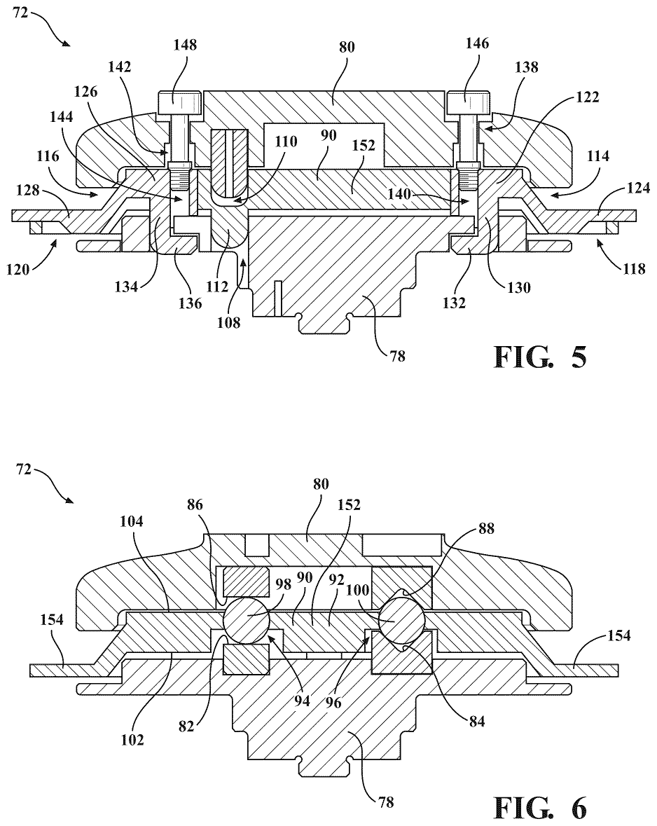

As shown in FIG. 5, the first mount component 78 may further include one chosen from a kinematic post 106 and a kinematic slot 108, and the second mount component 80 may further include the other of the kinematic post and the kinematic slot 108. Said differently, the first mount component 78 may include the kinematic post 106 and the second mount component 80 may include the kinematic slot 108, or the first mount component 78 may include the kinematic slot 108 and the second mount component 80 may include the kinematic post 106. The kinematic post 106 is engageable with the kinematic slot 108 to constrain one degree of freedom of movement between the first mount component 78 and the second mount component 80. Moreover, engagement of the first elongated kinematic coupler 98 with the first elongated mount surface 82 and the third elongated mount surface 86, engagement of the second elongated kinematic coupler 100 with the second elongated mount surface 84 and the fourth elongated mount surface 88, and engagement of the kinematic post 106 with the kinematic slot 108 may collectively constrain six degrees of freedom of movement between the first mount component 78 and the second mount component 80. Although not required, it is to be appreciated that each of the six degrees of freedom of movement constrained between the first mount component 78 and the second mount component 80 may only be constrained once; in other words, there may be no redundant constraints on any particular degree of freedom.

The kinematic post 106 may be engageable with the kinematic slot 108 through direct contact therebetween. In a non-limiting example, the kinematic post 106 may extend through the interface body 92 and into the kinematic slot 108. However, the kinematic post 106 need not be in direct contact with the kinematic slot 108 to be engageable with the kinematic slot 108. The kinematic post 106 may be engageable with the kinematic slot 108 without direct contact therebetween. As such, it is to be appreciated that the kinematic post 106 may be engageable with the kinematic slot 108 without breaking any sterile barrier formed in whole or in part by the interface component 90. Although not required, the interface component 90 may define an intermediate kinematic slot 110 engageable with the kinematic post 106, and the interface component 90 may have an intermediate kinematic post 112 engageable with the kinematic slot 108. Thus, through engagement of the kinematic post 106 with the intermediate kinematic slot 110 and engagement of the intermediate kinematic post 112 with the kinematic slot 108, one degree of freedom of movement between the first mount component 78 and the second mount component 80 may be constrained without breaking a sterile barrier established in whole or in part by the interface component 90. Additionally, the kinematic slot 108 may be aligned with the intermediate kinematic slot 110, and the kinematic post 106 may be aligned with the intermediate kinematic post 112, thus saving space within the kinematic mounting assembly 72.

As shown in FIGS. 2, 4, and 5, the interface component 90 may be retained to the first mount component 78. In a non-limiting example, the interface component 90 is fixed to the first mount component 78. The first mount component 78 may define a first notch 114 and the first mount component may also define a second notch 116 spaced from the first notch 114. The interface component 90 may include a first resilient insert 118 disposable in the first notch 114 and a second resilient insert 120 disposable in the second notch 116 such that the interface component 90 is detachably coupled to the first mount component 78. The first resilient insert 118 may have a first proximate portion 122 and a first distal portion 124 extending distally away from the first proximate portion 122, and the second resilient insert 120 may have a second proximate portion 126 and a second distal portion 128 extending away from the second proximate portion 126. The first proximate portion 122 may extend approximately parallel to the first mount component 78, and the second proximate portion 126 may extend approximately parallel to the first mount component 78. The first distal portion 124 may be angled to extend toward the first mount component 78 and the second distal portion 128 may be angled to extend toward the first mount component 78, as shown in FIG. 5.

The first resilient insert 118 may include a first tab 130 extending from the first proximate portion 122 toward the first mount component 78 and into the first notch 114. The first tab 130 may have a first lip 132 extending proximally from the first tab 130, and the first lip 132 may be engageable with the first mount component 78 to prevent the first resilient insert 118 from moving away from the first mount component 78. The second resilient insert 120 may include a second tab 134 extending from the second proximate portion 126 toward the second mount component 80 and into the second notch 116. The second tab 134 may have a second lip 136 extending proximally from the second tab 134. The second lip 136 may be engageable with the first mount component 78 to prevent the second resilient insert 120 from moving away from the first mount component 78. Thus, the first resilient insert 118 and the second resilient insert 120, and more specifically the first tab 130, the first lip 132, the second tab 134, and the second tab 134, may assist in ensuring that the interface component 90 is retained to the first mount component 78.

The first distal portion 124 of the first resilient insert 118 may be moveable toward the second mount component 80 to move the first resilient insert 118 out of the first notch 114, and the second distal portion 128 of the second resilient insert 120 may be moveable toward the second mount component 80 to move the second resilient insert 120 out of the second notch 116, thus detaching the interface component 90 from the first mount component 78. More specifically, moving the first distal portion 124 of the first resilient insert 118 toward the second mount component 80 may disengage the first tab 130, and particularly the first lip 132 of the first tab 130, from the first mount component 78 such that the first resilient insert 118 may be moved out of the first notch 114. Similarly, moving the second distal portion 128 of the second resilient insert 120 toward the second mount component 80 may disengage the second tab 134, and particularly the second lip 136 of the second tab 134, from the first mount component 78 such that the second resilient insert 120 may be moved out of the second notch 116.

The second mount component 80 may be retained to the interface component 90, the first mount component 78, or to both the interface component 90 and the first mount component 78. In a non-limiting example, the second mount component 80 is fixed to the interface component 90, the first mount component 78, or to both the interface component 90 and the first mount component 78. As shown in FIG. 5, the second mount component 80 may define a first bore 138, and the first proximate portion 122 of the first resilient insert 118 may define a first threaded bore 140 aligned with the first bore 138 of the second mount component 80. The second mount component 80 may also define a second bore 142, and the second proximate portion 126 of the second resilient insert 120 may define a second threaded bore 144 aligned with the second bore 142 of the second mount component 80. Although the first threaded bore 140 and the second threaded bore 144 are shown in FIG. 5 as extending completely through the first resilient insert 118 and the second resilient insert 120, respectively, it is to be appreciated that the first threaded bore 140 may be defined to not extend completely through the first resilient insert 118 and the second threaded bore 144 may be defined to not extend completely through the second resilient insert 120. In other words, the first threaded bore 140 and the second threaded bore 144 may be blind holes. As such, any sterile barrier created between the first mount component 78 and the second mount component 80 may be maintained. Alternatively, it is to be appreciated that a first plug may be inserted in the first threaded bore 140 and a second plug may be inserted in the second threaded bore 144 to ensure that any sterile barrier created between the first mount component 78 and the second mount component 80 is maintained. The first plug and the second plug permit the first threaded bore 140 and the second threaded bore 144 to be machined completely through the first resilient insert 118 and the second resilient insert 120, respectively, thus saving manufacturing time. The kinematic mounting assembly 72 may further include a first fastener 146 disposed in the first bore 138 and in the first threaded bore 140 and may also further include a second fastener 148 disposed in the second bore 142 and in the second threaded bore 144 to fix the second mount component 80 to the interface component 90. It is to be appreciated that the first fastener 146 and the second fastener 148 may be a bolt, a screw, or the like. The first fastener 146 and the second fastener 148 may both be threaded to engage the first threaded bore 140 and the second threaded bore 144, respectively.

Although not required, the kinematic mounting assembly 72 may further include a drape 150 configured to cooperate with the interface component 90 to form a sterile barrier to the first mount component 78, as shown in FIG. 1. It is to be appreciated, therefore, that the interface component 90 may be sterilizable. The interface component 90 may be formed from a metal, a metal alloy, sterilizable plastic, or sterilizable composite. The interface component 90 and the drape 150 may permit the second mount component 80 to be detached from the interface component 90 without breaking the sterile barrier to the first mount component 78. It is to be appreciated, therefore, that the first mount component 78 and the interface component 90 may be together configured to permit the drape 150 to be positioned such that a sterile barrier to the first mount component 78 is formed by the interface component 90 and the drape 150.

Moreover, it is also to be appreciated that the second mount component 80 may be detachable from the interface component 90 without breaking a sterile barrier to the first mount component 78. More specifically, the interface body 92 may include a central hub 152 defining the first elongated opening 94 and the second elongated opening 96. The interface body 92 may also include an outer flange 154 disposed about the central hub 152. The outer flange 154 is configured to cooperate with a drape 150 such that the interface component 90 and the drape 150 form a sterile barrier to the first mount component 78. In a non-limiting example, the outer flange 154 and/or the first resilient insert 118, particularly the first distal portion 124, and/or the second resilient insert 120, particularly the second distal portion 128, may be configured to snap into the drape 150 such that the interface component 90 and the drape 150 form a sterile barrier to the first mount component 78. In another non-limiting example, the drape 150 may include an adhesive which forms a temporary bond to the outer flange 154 to form a sterile barrier to the first mount component 78. The drape 150 may be sterile, and may include a flexible sheet material defining a hole and an attachment element surrounding the hole. The first mount component 78, specifically the first elongated mount surface 82 and the second elongated mount surface 84, may be accessible through the hole in the drape 150 to permit attachment of the interface component 90 to the first mount component 78 while the first mount component 78 is draped.

Thus, the second surgical component 76 (when coupled to the second mount component 80) may also be detachable from the interface component 90 without breaking a sterile barrier to the first mount component 78. In the implementations where the second surgical component 76 is the surgical end effector 26, the surgical end effector 26 may be replaced with another surgical end effector without breaking the sterile barrier. In this way, a surgeon may swap surgical end effectors 26 during the surgical procedure without risking breaking the sterile barrier and without risking infection to the patient, permitting the surgeon to access multiple different surgical end effectors 26, potentially having different functionalities, during the surgical procedure.

In a second aspect, an interface component 90 is provided. The interface component 90 is disposable between a first mount component 78 and a second mount component 80 in a kinematic mounting assembly 72. The interface component 90 includes an interface body. The interface body 92 has a first interface surface 102 configured to interface with the first mount component 78 and has a second interface surface 104 configured to interface with the second mount component 80. The interface body 92 may define a first elongated opening 94 extending from the first interface surface 102 to the second interface surface 104, and may also define a second elongated opening 96 extending from the first interface surface 102 to the second interface surface 104. The interface component 90 also includes a first elongated kinematic coupler 98 having an elongated shape disposed in the first elongated opening 94 and moveable within the first elongated opening 94 relative to the interface body 92. The interface component 90 further includes a second elongated kinematic coupler 100 having an elongated shape disposed in the second elongated opening 96 and moveable within the second elongated opening 96 relative to the interface body 92.

In a third aspect, a kinematic mounting assembly 72 for coupling a first surgical component 74 and a second surgical component 76 is provided. The kinematic mounting assembly 72 includes a first mount component 78 couplable to the first surgical component 74. The first mount component 78 includes a first kinematic mount surface 82 having a first elongated shape and a second kinematic mount surface 84 having a second elongated shape different from the first elongated shape. The first kinematic mount surface 82 and the second kinematic mount surface 84 may extend approximately parallel to one another. The kinematic mounting assembly 72 also includes a second mount component 80 couplable to the second surgical component 76. The second mount component 80 includes a third kinematic mount surface 86 having a third elongated shape and a fourth kinematic mount surface 88 having a fourth elongated shape different from the third elongated shape. The third kinematic mount surface 86 and the fourth kinematic mount surface 88 may extend approximately parallel to one another. The kinematic mounting assembly 72 further includes an interface component 90 disposed between the first mount component 78 and the second mount component 80. The interface component 90 includes a first kinematic coupler 98 having a fifth elongated shape. The first kinematic coupler 98 is engageable with the first kinematic mount surface 82 and the third kinematic mount surface 86. The interface component 90 also includes a second kinematic coupler 100 having the fifth elongated shape. The second kinematic coupler 100 is engageable with the second kinematic mount surface 84 and the fourth kinematic mount surface 88. The first kinematic coupler 98 and the second kinematic coupler 100 may extend approximately parallel to one another.

In a fourth aspect, a mounting system 72 for coupling first and second surgical components 74, 76 is provided. The mounting system 72 includes a first mounting portion 78 coupled to the first surgical component 74. The first mounting portion 78 includes a first pair of kinematic mounting surfaces 82, 84 having different shapes as one another and extending approximately parallel to one another. The first mounting portion 78 also includes a kinematic post 106 or a kinematic slot 108. The mounting system 72 also includes a second mounting portion 80 coupled to the second surgical component 76. The second mounting portion 80 includes a second pair of kinematic mounting surfaces 86, 88 having different shapes as one another and extending approximately parallel to one another. The second mounting portion 80 also includes the other of the kinematic post 106 or the kinematic slot 108. The mounting system 72 further includes a barrier portion 90 configured to be disposed between the first and second mounting portion 78, 80. The barrier portion 90 includes a pair of kinematic couplers 98, 100 having identical shapes as one another and extending approximately parallel to one another. When the portions 78, 80, 90 are secured together, the pair of kinematic couplers 98, 100 are configured to engage the first and second pair of kinematic mounting surfaces 82, 84, 86, 88 and the kinematic post 106 is configured to engage the kinematic slot 108 to collectively constrain six degrees of freedom of movement between the first and second surgical components 74, 76.

The invention has been described in an illustrative manner, and it is to be understood that the terminology which has been used is intended to be in the nature of words of description rather than of limitation. Many modifications and variations of the present invention are possible in light of the above teachings, and the invention may be practiced otherwise than as specifically described.

What is claimed is:

1. A kinematic mounting assembly for coupling a first surgical component and a second surgical component, the kinematic mounting assembly comprising:
a first mount component couplable to the first surgical component, with the first mount component comprising:
a first elongated mount surface, and
a second elongated mount surface spaced relative to the first elongated mount surface;
a second mount component couplable to the second surgical component, with the second mount component comprising:
a third elongated mount surface, and
a fourth elongated mount surface spaced relative to the third elongated mount surface; and
an interface component disposed between the first mount component and the second mount component, the interface component comprising:
an interface body,
a first elongated kinematic coupler coupled to the interface body and engageable with the first elongated mount surface and the third elongated mount surface, and
a second elongated kinematic coupler coupled to the interface body and engageable with the second elongated mount surface and the fourth elongated mount surface.

2. The kinematic mounting assembly of claim 1, wherein the first elongated mount surface extends approximately parallel to the second elongated mount surface, wherein the third elongated mount surface extends approximately parallel to the fourth elongated mount surface, and wherein the first elongated kinematic coupler extends approximately parallel to the second elongated kinematic coupler.

3. The kinematic mounting assembly of claim 1, wherein:
the interface body defines a first elongated opening and a second elongated opening spaced from the first elongated opening;
the first elongated kinematic coupler is disposed in the first elongated opening and moveable within the first elongated opening relative to the interface body; and
the second elongated kinematic coupler is disposed in the second elongated opening and moveable within the second elongated opening relative to the interface body.

4. The kinematic mounting assembly of claim 3, wherein the first elongated opening and the second elongated opening are defined approximately parallel to one another.

5. The kinematic mounting assembly of claims 3, wherein the first elongated kinematic coupler is resiliently fixed to the interface body within the first elongated opening, and wherein the second elongated kinematic coupler is resiliently fixed to the interface body within the second elongated opening.

6. The kinematic mounting assembly of claim 1, wherein engagement of the first elongated kinematic coupler with the first elongated mount surface and the third elongated mount surface and engagement of the second elongated kinematic coupler with the second elongated mount surface and the fourth elongated mount surface collectively constrains five degrees of freedom of movement between the first mount component and the second mount component.

7. The kinematic mounting assembly of claim 1, wherein the first elongated mount surface has a first elongated shape, and wherein the second elongated mount surface has a second elongated shape different from the first elongated shape.

8. The kinematic mounting assembly of claim 7, wherein the third elongated mount surface has the first elongated shape, and wherein the fourth elongated mount surface has the second elongated shape.

9. The kinematic mounting assembly of claim 8, wherein the first elongated shape of the first elongated mount surface and the third elongated mount surface is planar such that one rotational degree of freedom of movement is constrained between the first mount component and the second mount component upon engagement of the first elongated kinematic coupler with the first elongated mount surface and the third elongated mount surface.

10. The kinematic mounting assembly of claim 8, wherein the second elongated shape of the second elongated mount surface and the fourth elongated mount surface is a concave groove.

11. The kinematic mounting assembly of claim 1, wherein the first elongated kinematic coupler is cylindrical and the second elongated kinematic coupler is cylindrical.

12. The kinematic mounting assembly of claim 3, wherein the interface body has a first interface surface facing the first mount component to interface with the first mount component, wherein the interface body has a second interface surface facing the second mount component to interface with the second mount component, wherein the first elongated opening is defined to extend from the first interface surface through the interface body to the second interface surface, and wherein the second elongated opening is defined to extend from the first interface surface through the interface body to the second interface surface.

13. The kinematic mounting assembly of claim 1, wherein the first mount component further comprises one chosen from a kinematic post and a kinematic slot, wherein the second mount component further comprises the other of the kinematic post and the kinematic slot, and wherein the kinematic post is engageable with the kinematic slot to constrain one degree of freedom of movement between the first mount component and the second mount component.

14. The kinematic mounting assembly of claim 13, wherein the interface component defines an intermediate kinematic slot engageable with the kinematic post, and wherein the interface component has an intermediate kinematic post engageable with the kinematic slot.

15. The kinematic mounting assembly of claim 1, wherein the first mount component defines a first notch and a second notch spaced from the first notch, and wherein the interface component includes a first resilient insert disposable in the first notch and a second resilient insert disposable in the second notch such that the interface component is detachably coupled to the first mount component.

16. The kinematic mounting assembly of claim 15, wherein the first resilient insert has a first proximate portion and a first distal portion extending distally away from the first proximate portion, wherein the second resilient insert has a second proximate portion and a second distal portion extending away from the second proximate portion, wherein the first distal portion is angled to extend toward the first mount component, and wherein the second distal portion is angled to extend toward the first mount component.

17. The kinematic mounting assembly of claim 1 further comprising a drape configured to cooperate with the interface component to form a sterile barrier to the first mount component, and wherein the interface component and the drape permit the second mount component to be detached from the interface component without breaking the sterile barrier to the first mount component.

18. The kinematic mounting assembly of claim 1, wherein the first mount component and the interface component are together configured to permit a drape to be positioned such that a sterile barrier to the first mount component is formed by the interface component and the drape and such that the second mount component is detachable from the interface component without breaking the sterile barrier to the first mount component.

19. The kinematic mounting assembly of claim 3, wherein the interface body includes a central hub defining the first elongated opening and the second elongated opening and an outer flange disposed about the central hub, with the outer flange configured to cooperate with a drape such that the interface component and the drape form a sterile barrier to the first mount component.

20. A robotic surgical system, comprising:
a robotic arm having a distal flange;
a surgical end effector; and
a kinematic mounting assembly coupled to the distal flange of the robotic arm and coupled to the surgical end effector, with the kinematic mounting assembly comprising:
a first mount component coupled to the distal flange of the robotic arm, with the first mount component comprising:
a first elongated mount surface, and
a second elongated mount surface spaced relative to the first elongated mount surface;
a second mount component coupled to the surgical end effector, with the second mount component comprising:
a third elongated mount surface, and
a fourth elongated mount surface spaced relative to the third elongated mount surface; and
an interface component disposed between the first mount component and the second mount component, the interface component comprising:
an interface body,
a first elongated kinematic coupler coupled to the interface body and engageable with the first elongated mount surface and the third elongated mount surface, and
a second elongated kinematic coupler coupled to the interface body and engageable with the second elongated mount surface and the fourth elongated mount surface.

21. An interface component disposable between a first mount component and a second mount component in a kinematic mounting assembly, the interface component comprising:
an interface body including a first interface surface configured to interface with the first mount component and a second interface surface configured to interface with the second mount component;
a first elongated kinematic coupler having an elongated shape and being coupled to the interface body and moveable relative to the interface body; and
a second elongated kinematic coupler having an elongated shape and being coupled to the interface body and moveable relative to the interface body.
